(19) United States
(12) Patent Application Publication
(10) Pub. No.: US 2008/0187922 A1
(43) Pub. Date: Aug. 7, 2008

(54) METHOD OF SCREENING DRUG-RESISTANCE PROTEIN OF MYCOBACTERIUM TUBERCULOSIS

(75) Inventors: Honghai Wang, Shanghai (CN); Lu Zhang, Shanghai (CN); Qingzhong Wang, Shanghai (CN); Jun Yue, Shanghai (CN); Min Zhang, Shanghai (CN); Ying Xu, Shanghai (CN); Bingdong Zhu, Shanghai (CN); Jiuling Wang, Shanghai (CN); Yaqing Qie, Shanghai (CN)

Correspondence Address: LADAS & PARRY, 5670 WILSHIRE BOULEVARD, SUITE 2100, LOS ANGELES, CA, 90036-5679, US

(73) Assignee: FuDan University, Shanghai (CN)

(21) Appl. No.: 11/871,932

(22) Filed: Oct. 12, 2007

(30) Foreign Application Priority Data
Oct. 19, 2006 (CN) 200610117294.1
Oct. 30, 2006 (CN) 200610117754.0

(51) Int. Cl. C12Q 1/68

(52) U.S. Cl. 435/6

(57) ABSTRACT

The present invention belongs to the field of molecular biology. Specially, the invention relates to the screenings of a new drug target, a vaccine antigen and a test target of anti-tuberculosis treatment. Specially, based on the differential proteome technology and by the techniques of gene sequencing, in vitro protein expression, immunological detection and gene recombination, the invention primarily certifies that the up-regulated expression of Protein Rv2629 and the mutation of Gene 191A/C of Mycobacterium tuberculosis are related to the Rifampicin (RFP) drug-resistance, which provides useful information for the drug-resistance detection of Mycobacterium tuberculosis, the novel drug design and the vaccine development. Firstly, the drug-resistant strains are cultured and the proteins from drug resistance strains and sensitive strains are isolated respectively. Secondly, the proteins from the drug resistance strains and the sensitive strains are compared to determine the differential protein sites, such that the drug-resistant proteins are identified by means of Mass Spectrometry. Next, the up-regulated expression of Protein Rv2629 and the mutation of gene are correlated to the drug-resistance of Mycobacterium tuberculosis by means of gene sequencing technique. Finally, after the gene cloning, the in vitro expression and purification of the protein, the wild-type and mutant-type recombinant protein of Rv2629 are obtained. The polyclonal antibody is prepared, and the protein subcellular localization is carried out. The Minimum Inhibitory Concentrations (MICs) of the strains containing different plasmids obtained by the transformation of recombinant shuttle plasmids are also measured. The gene encoding the protein Rv2629 in the genome of Mycobacterium tuberculosis is isolated and is up-regulation expressed in a Rifampicin (RFP)-resistant strains. And the A/C gene mutation at nucleotide 191 which is related to drug-resistance is detected. The relationship between the mutation and the Rifampicin (RFP)-resistance is primarily certified by molecular biology technique and the in vitro experiments. This invention provides methods used to accurately isolate and identify the drug-resistance protein of Mycobacterium tuberculosis. The A/C mutation of Rv2629 at nucleotide 191 related to the drug-resistance of Mycobacterium tuberculosis is isolated, which shows new approach for the further study of Mycobacterium tuberculosis drug-resistant mechanism, the rapid detection of clinical drug-resistance strains, as well as the developments of new drug targets and vaccines.

### FIELD OF THE INVENTION

The present invention belongs to the field of molecular biology. Specially, the invention relates to the screening of new drug targets, new vaccine antigens and new diagnostic markers of anti-tuberculosis treatment. More specially, the invention provides a method of screening the Rifampicin (RFP)-resistance protein of Mycobacterium tuberculosis via Proteomics technology. The present invention also provides the uses of this method in the screening of drugs and in the preparation of vaccines.

### DESCRIPTION OF THE PRIOR ART

Tuberculosis (TB) is a tremendous threat to the human health. At present nearly 32% of the global populations are infected by Mycobacterium tuberculosis (M.tb). Tuberculosis kills about 2.7 million people every year, that is, the daily mortality is more than 7000. Accordingly, the World Health Organization (WHO) declared TB as a global health emergency in 1993.

China is one of the 22 countries subject to a high TB burden in the world, and ranks the second in terms of total number of TB patients. Based on the investigation report of 4th National Epidemiological Sampling Survey of Tuberculosis in 2000, the epidemic situations is very serious in China, which just only follows India in the world. 55 million persons have been infected by Mycobacterium tuberculosis in China, including 4.51 million active TB subjects, and 2 million of them are infectious. Every year 130 thousand persons die of TB, thus TB has been the first killer among all the infectious diseases in China.

In March 2000, WHO and IUATLD (International Union Against Tuberculosis and Lung Disease) reported the surveillance results of drug-resistance tuberculosis in 72 countries and regions of the world, in which the average drug-resistance rate against at least one kind of antituberculotics was 10.7% (1.7%˜36.9%). Among the 550 million persons infected by Mycobacterium Tuberculosis in China, the total drug resistance rate is 27.8%, the initial drug resistance rate is 18.6%, the acquired drug resistance rate is 46.5%˜68.4%, the total multi-drug resistance is 10.7%, and the number of drug-resistance TB patients is 555 thousand at present. The long periods of drug-resistance tuberculosis diagnosis and traditional drug sensitive experiment result in that the subjects could not be treated and isolated in time, additionally, the conventional antituberculotics are not available, which results in that the period of treatment extends and disease infectivity increases, which make the drug-resistance patients die or become main infection source. Meanwhile, the cost on drug-resistance tuberculosis increases by more than 50 times, which wastes a large amount of health resources.

The occurrence and the spread of drug-resistance TB as well as the epidemic of AIDS are the main reasons of the current epidemic of TB, which has been a serious problem of global public health. It can be predicted that the TB epidemic situation is much more serious in 21^{st} century than in the mid 20^{th} century.

Rifampicin (RFP, 3-(4-Methylpiperazinyliminomethyl)-rifamycin) has been used as an antituberculotic since 1972. RFP can specifically bind to the β-subunit of RNA polymerase so as to inhibit RNA polymerase's bioactivity, which interferes the transcription and synthesis of RNA of Mycobacterium so as to hinder its protein synthesis and consequently exert an antibiotic action. Since RFP has a minimal inhibition concentrations (MICs) of 0.1˜0.2 μg/mL, it is enough to kill M.tb in living state. Therefore, RFP is one of the two main antituberculotics in addition to Isoniazid, and it is able to significantly shorten the course of TB treatment. The mutation of gene rpoB encoding the β-subunit of RNA polymerase is the molecular mechanism of more than 90% RFP-resistance strains. It is found that rpoB mutation does not occurs in a few RFP-resistance strains, on the other hand rpoB mutation can be detected in a few RFP sensitive strains, which indicates that there is other drug-resistance mechanisms. It is also found that the RFP-resistance strains are usually resistant to other antituberculotic. Therefore, the study on the RFP-resistance strains is significant.

Proteome technique makes it possible to represent the proteome characteristics in a high-throughput way. With the approach of comparative proteome, the pathogenic factors and pathogenic mechanisms of pathogenic bacteria can be determined quickly, such that a novel vaccine can be configured, which facilitates the developments of new anti-pathogen drugs and diagnostic reagents. Furthermore, the nucleotide sequence encoding protein Rv2629 in the M.tb genome is isolated by the combination with the gene sequencing technique. Said Rv2629 protein not only is up-regulation expressed in RFP-resistance strains, but also codes gene mutates at the site of 191 A/C, which is highly correlated with RFP-resistance. The correlation between the A/C mutation and RFP-resistance has been preliminarily identified via in vitro experiments by means of molecular biological technique.

Presently, protein expression of Mycobacterium complex strains has been studied by comparative proteomics technique, and gene expression of Mycobacterium tuberculosis (M.tb) in multiple conditions has been analyzed. However, the comparison analysis of the whole cell proteins of therapeutic drug-resistance M.tb has not been reported yet.

### SUMMARY OF THE INVENTION

One purpose of the present invention is to provide a method of screening the RFP-resistance protein of Mycobacterium tuberculosis.

Another purpose of the invention is to provide database of drug-resistance genes and proteins, which can provide drug targets for the development of new antituberculotics. The invention also provides a gene bank and a protein bank so as to facilitate the development of techniques used to detect clinical drug-resistance strains. Moreover, the present invention provides databases of candidative genes and proteins so as to facilitate the construction of new therapeutic vaccines.

Another purpose of the invention is to provide mutated genes of RFP-resistance. It has been identified that A/C mutation at nucleotide 191 of gene Rv2629 relates to the drug-resistance of M.tb by means of mass gene sequencing technique.

Another purpose of the invention is to subcellularly localize protein Rv2629 at the wall and the membrane of M.tb.

A still another purpose of the invention is to provide in vitro experiments, in which the correlation between A/C mutation at nucleotide 191 of gene Rv2629 and RFP-resistance are preliminarily identified by gene knock-in technique.

The invention also provides a method for screening the drug-resistance proteins of Mycobacterium tuberculosis resistant to the drug Rifampicin (RFP, 3-(4-Methylpiperazinyliminomethyl)-rifamycin), including the following steps:

(1) Culturing M.tb strains under the same conditions with drug or without drug, and defining a strain having a MIC of 16 μg/mL as a drug-resistance strain;

(2) Separating proteins from drug-resistance strains and drug sensitive strains respectively;

(3) Comparing the proteins of a drug-resistance strain with that of a sensitive strain and defining a protein additive or absent in the drug-resistance strain or a protein of which the expression increases by at least twice or more, or a protein of which the expression reduces by at least a half less as a drug-resistance;

(4) Separating and identifying the drug-resistance protein;

(5) Extracting the genome of a M.tb strain, and amplifying the target gene by PCR by using a configured gene sequencing primers, and then analyzing gene sequence by automatic sequenator, followed by screening the mutated genes related to the drug-resistance of M.tb;

(6) Constructing plasmids pET30 and pMV261 by gene cloning technique, and then transforming the wild-type or mutant-type Rv2629 genes into a E. coli or Mycobacterium smegmatis strain, then detecting the RFP MICs values of the Mycobacterium smegmatis recombinant strain containing a wild-type (or a mutant-type) pMV261::Rv2629 gene, respectively;

(7) In vitro preparing the recombinant protein respectively, and preparing polyclonal antibody, and then subcellularly localizing the protein Rv2629 in M.tb by Western-blot.

In the step (2) of the invention, only the basic proteins in the drug-resistance strains and sensitive strains are retaining.

During the period of MIC detection in the step (1) and the period of protein isolation in the step (2), the strains used might be M.tb strains in the logarithmic growth phase.

In the steps (2) and (4) of the invention, the protein could be separated by electrophoresis, centrifugal, salting-out, filtration or chromatography. All experiments are carried out by conventional operation methods and under normal conditions except for special explanation.

In the step (4), the protein could be identified by Electrophoregram Analysis, protein sequencing, mass spectrum analysis or amino acid composition analysis.

In the step (4), the resultant drug-resistance protein could be used as a drug target for screening or detecting antituberculotics. The principle of screening and detecting antituberculotics lies in that a substance capable of combining and interacting with a drug-resistance protein or affecting the expression of the protein could be used as a candidative antituberculotic. The structures of the drug-resistance protein and said substance can be simulated by the bioinformatics methods, thus a substance capable of combining and interacting with a drug-resistance protein could be isolated. Then the action site and mechanism between them could be further determined. The result is certified by experimental strains or animals.

In the step (4), the gene encoding the drug-resistance protein can be mutated to construct vaccines. After the protein is identified, the key amino acid residues could be further identified. Then, these residues could be mutated, and the corresponding mutant proteins or their encoding nucleotides could be used to construct vaccines.

In the step (5), most of RFP-resistant gene mutations are related to the polymorphism of M.tb, and the A/C mutant at nucleotide 191 of gene Rv2629 has possible association with the RFP-resistance of M.tb.

In the step (6), the four engineering bacteria pET30::Rv2629, pET30::Rv2629M (mutant-type, A/C mutant at nucleotide 191 of gene Rv2629, represented as the same hereafter), pMV261::Rv2629 and pMV261::Rv2629M are provided, and their sequences are confirmed to be correct after plasmid sequencing analysis. The RFP MICs of the resultant Mycobacterium smegmatis recombinant strains containing pMV261::Rv2629 and pET30::Rv2629M are 30 μg/mL and 150 μg/mL respectively.

In the step (7), the recombinant proteins Rv2629 and Rv2629M are obtained, and the resultant immunological titer of the polyclonal antibodies is more than 12,800 (examined by ELISA). The Western-blot result shows that the protein Rv2629 mainly exists in the culture medium filtration proteins of M.tb, the cell wall and the cell membrane.

The cellular proteins of clinical RFP-resistance strains are firstly systematically analyzed by comparative proteome technique in the present invention. Meanwhile, by using a human experimental standard strain H37Rv and a clinical sensitive strain as controls, 5 clinical RFP-resistant strains, 9 clinical sensitive strains and 1 standard strain H37Rv are detected, and each of the 15 M.tb samples has been tested at least three times. Among the 13 resultant drug-resistance related proteins, 6 proteins are up-regulated, they are trpC (Rv1611), wag31 (ag84, Rv2145c, R6), bfrB (Rv3841, R4), cysK1 (cysK, Rv2334, R8), Rv2629 (R9), ilvN (ilvH, Rv3002c, 10#), devR (dosR,Rv3133c, R11); and 7 proteins are down-regulated, they are Ssb (Rv0054, R1), Rv0068 (R2), Rv0927c (R3), OpcA (Rv1446c, R5), Rv2159 (7#), PPE51 (Rv3136, R12) and NuoB (Rv3146, 13#). The A→C mutant at nucleotide 191 of gene Rv2629 was identified by the technique of PCR and gene sequencing, which leads to a GAT→GCT gene mutant and an Asp→Ala amino acid variation. This kind of mutation just only appears in the clinical RFP-resistant strains, which is absent in the standard strain H37Rv and clinical sensitive strains. With further in vitro molecular biological experiments, it is shown that protein Rv2629 mainly subcellularly localized in the culture medium filtration proteins, the cell wall and the cell membrane of M.tb. With gene knock-in experiments, it is preliminarily demonstrated that only if a gene Rv2629 carrying a 191 C mutant site is transformed into a Mycobacterium smegmatis strains, could the receptor strain obtain the RFP-resistance, whereas the RFP-resistance is not found in a wild-type Rv2629. So far the corresponding studies on the drug-resistance mechanism of M.tb have not been reported. The methods provided by the present invention could facilitate the development of a rapid M.tb drug-resistance detection technique and provide new approaches used to further study on the M.tb drug-resistance mechanism so as to develop novel antituberculotics and vaccines. No the same studies have been reported yet.

TB is the first killer to human beings among all the infectious diseases. Due to the complicated processes of the drug-resistance TB diagnosis and the long period of traditional drug sensitive experiment, the subjects could not be the treated and isolated in time, which makes the drug-resistance patients die or become main infection source. The drug-resistance has made the cost on drug-resistance tuberculosis treatment increase by 50 times or more, which wastes a large amount of health resources and aggravates the TB patients and their families' burden. The methods provided by the present invention facilitate the accurate isolation and identification of M.tb drug-resistance proteins. It also provides new approaches used to further study on the M.tb drug-resistance mechanism, rapidly detect M.tb drug-resistance strains and develop novel antituberculotics and vaccines.

### BRIEF DESCRIPTION OF THE FIGURES

FIG. 1 shows the 2-DE map of the bacterial proteins from an M.tb H37Rv strain (pH4˜7). 2-DE means the two-dimensional electrophoresis; the signs R1 etc. mean the differential expression protein sites (represented as the same hereafter).

FIG. 2 shows the 2-DE map of the bacterial protein from an M.tb clinical sensitive strain (pH4˜7).

FIG. 3 shows the 2-DE map of the bacterial protein from an M.tb clinical RFP-resistant strain (pH4˜7).

FIG. 4 shows the comparison of the cutting photograms of the bacterial protein R4 from M.tb clinical RFP-resistant strain (pH4˜7). (a): the M.tb H37Rv; (b): the clinical sensitive strain; (c) the M.tb clinical RFP-resistant strain; (d) the comparison of the protein expression quantities.

FIG. 5 shows the comparison of the cutting photograms of the bacterial protein R6 from M.tb clinical RFP-resistant strain (pH4˜7). (a): M.tb H37Rv; (b): the clinical sensitive strain; (c) the M.tb clinical RFP-resistant strain; (d) the comparison of protein expression quantities.

FIG. 6 shows the 2-DE map of the basic bacterial protein from the M.tb H37Rv strain (pH7˜10).

FIG. 7 shows the 2-DE map of the basic bacterial protein from the M.tb clinical RFP-resistant strain (pH7˜10).

FIG. 8 shows the comparison of the cutting photograms of basic bacterial protein R2 from the M.tb clinical RFP-resistant strain (pH7˜10). (a): M.tb H37Rv; (b): the clinical sensitive strain; (c) the M.tb clinical RFP-resistant strain; (d) the comparison of protein expression quantities.

FIG. 9 shows the comparison of the cutting photograms of basic bacterial protein 7# from the M.tb clinical RFP-resistant strain (pH7˜10). (a): M.tb H37Rv; (b): the clinical sensitive strain; (c) the M.tb clinical RFP-resistant strain; (d) the comparison of protein expression quantities.

FIG. 10 shows the peptide mass fingerprinting of the point R4. The x-axis shows the charge-mass ratio (m/z), and the y-axis shows the protein quantity (% intensity). Parameters of the mass spectrometry: 4700 reflector spec #1 MC [BP=1500.8, 7048].

FIG. 11 shows the 1228.6 MSMS of the point R4 (MSMS means the tandem mass spectrometry, 1228.6 is the molecular weight of a polypeptide fragment identified by PMF MS, and the polypeptide fragment is then degraded into amino acids to subject a further MS analysis, such that its amino acid components and sequence are identified). The x-axis shows charge-mass ratio (m/z), and the y-axis shows the protein quantity (% intensity). Parameters of mass spectrometry: 4700 MS/MS Precursor 1228.67 spec #4 [BP=432.4, 110].

FIG. 12 shows the 1550.8 MSMS of the point R4. The x-axis shows charge-mass ratio (m/z), and the y-axis shows the protein quantity (% intensity). Parameters of mass spectrometry: 4700 MS/MS Precursor spec #1 MC [BP=1752, 185].

FIG. 13 shows the gene sequence of Rv2629.

FIG. 14 shows the double incision enzyme cutting map of the wild-type gene Rv2629 and the recombinant plasmid pET30 of 191 A/C mutant-type.

FIG. 15 shows the subcellular localization of protein Rv2629 in M.tb (CFP: culture medium filtration protein, CW: cell wall, CM: cell membrane, CP: cytoplasm).

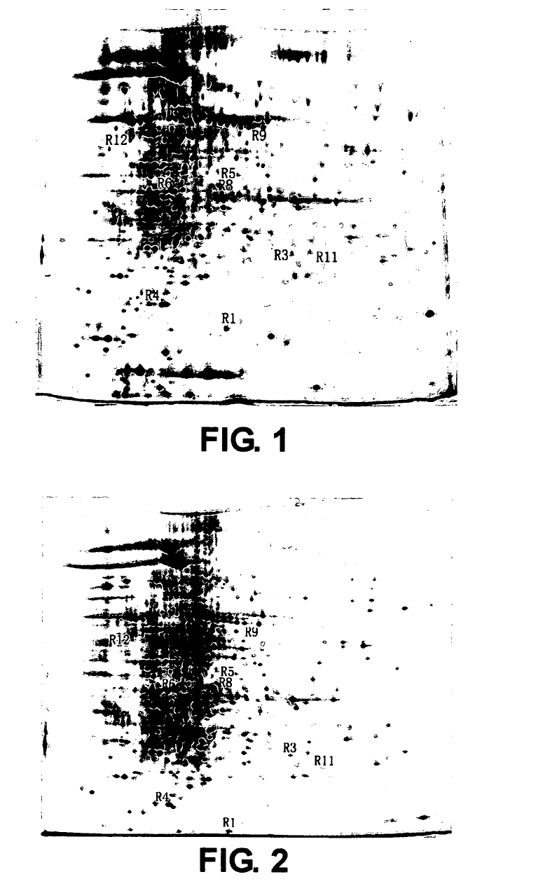

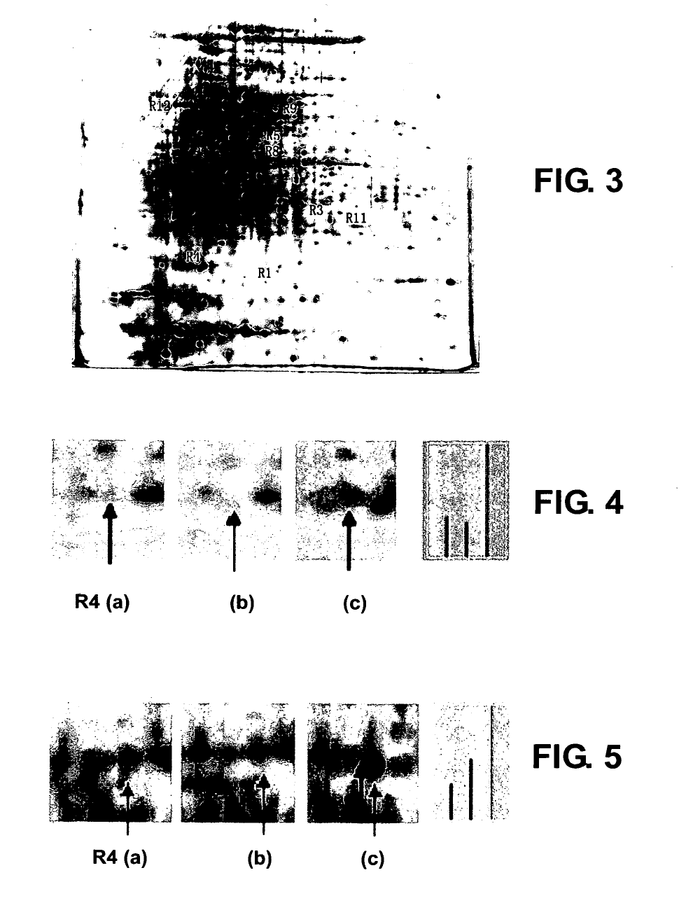

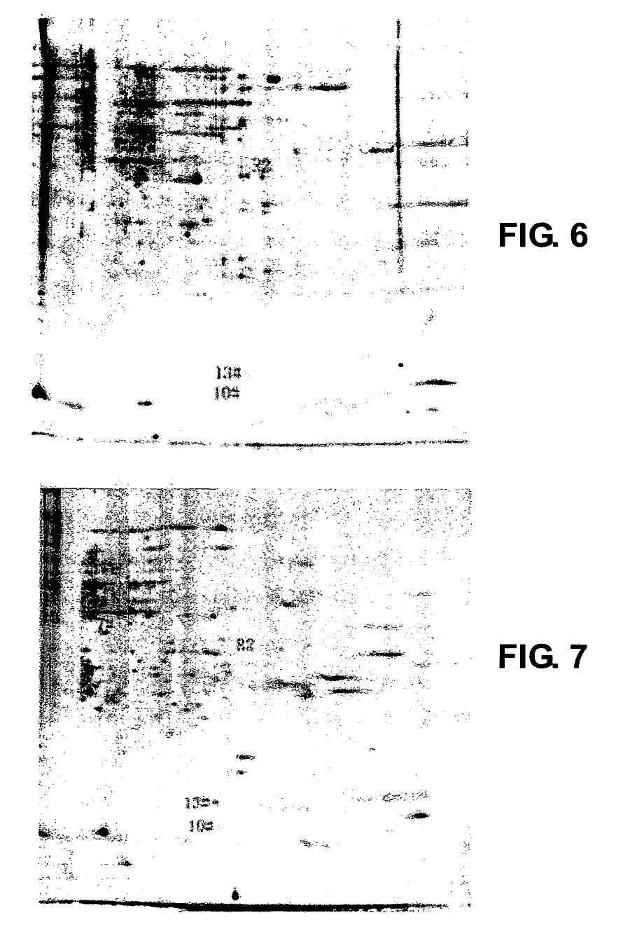

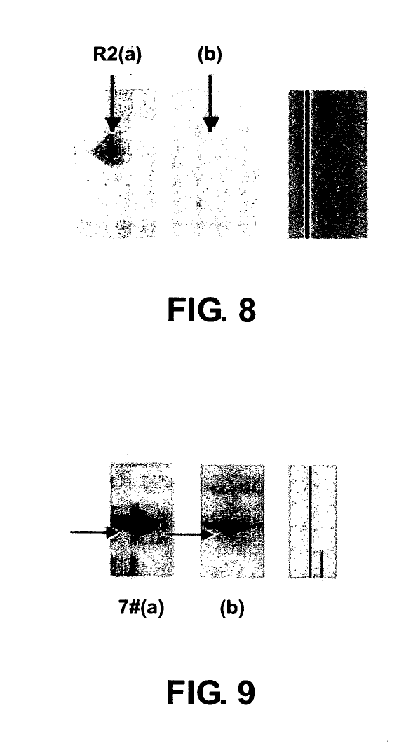

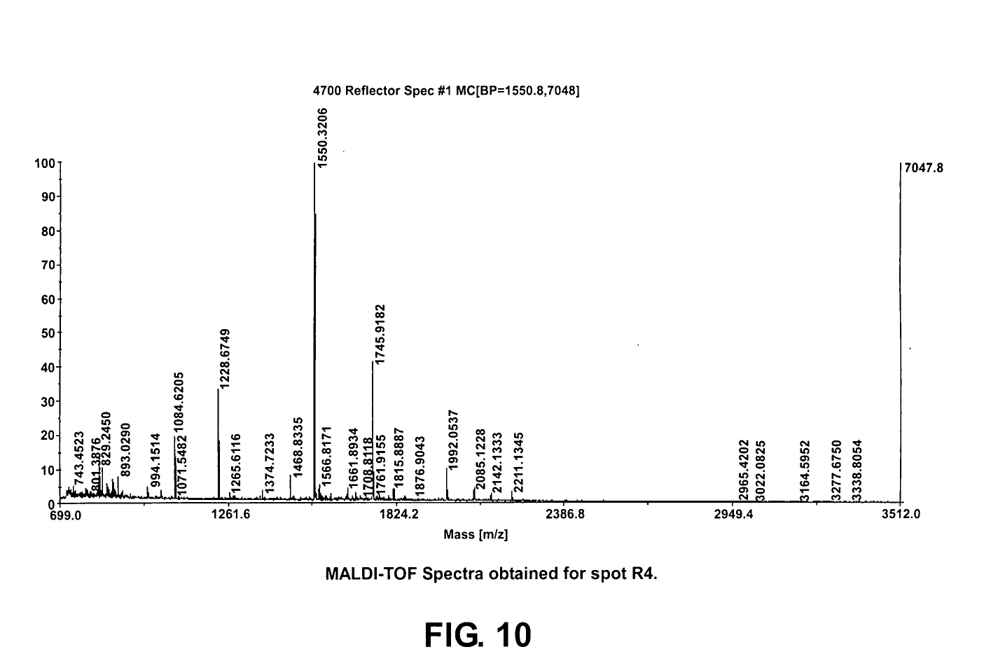

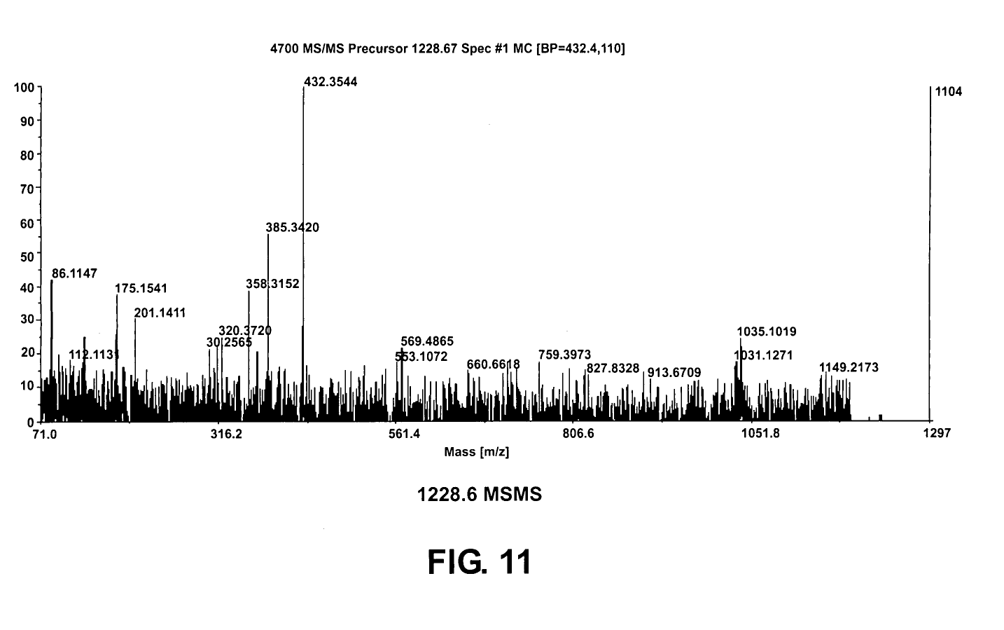

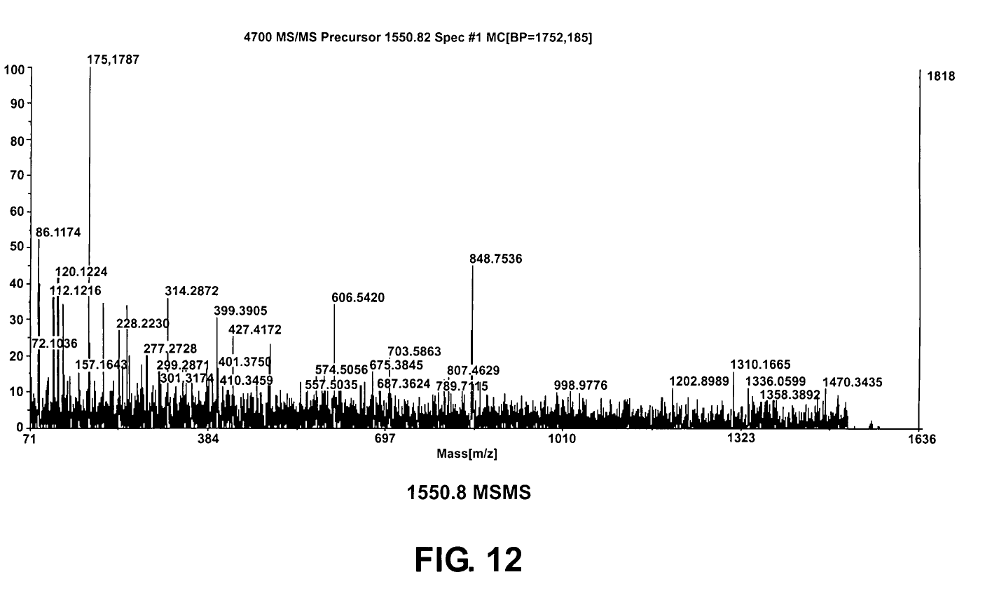

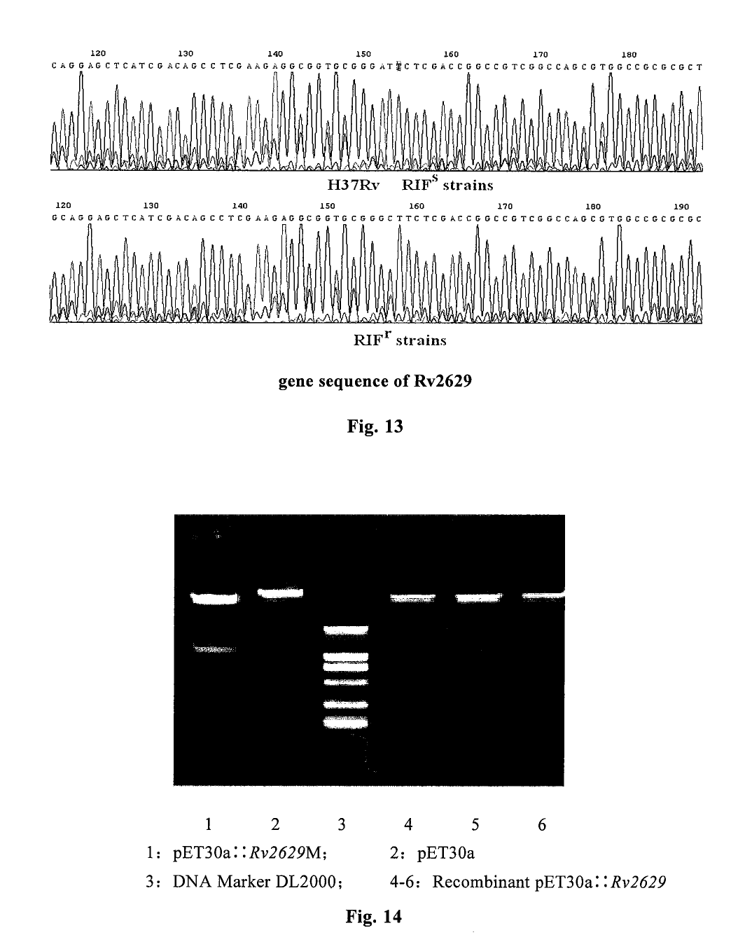

### DETAILED DESCRIPTION OF THE INVENTION

### EXAMPLE 1

### The Culture of M.tb and Drug Sensitive Experiments

The M.tb standard strain Mycobacterium tuberculosis H37Rv (ATCC93009), clinical RFP (Sigma, R3501)-resistant strains (5 strains), clinical sensitive strain (9 strains) are all supplied by Shanghai Pneumonoe Hospital TB Lab. Inoculate the strains into Middlebrook 7H9Broth (DIFCO, Bection-Dickinson) with 10% ADC (DIFCO, 0.5% BSA, 0.2% glucose, 140 mmol/L NaCl) and 5% glycerin, and then statically cultured for 15˜20 days in a 37 constant temperature incubator. The bacterium are collected when the bacterial concentration reaches a bacterial density of 1×10⁸˜2×10⁸ or OD₆₀₀ 0.8˜1.

The bacterium is killed in a water bath at 80 for 2 h, and the supernatant is removed after centrifugation at 5000 rpm, 4 (autoclaving at 121 for 30 min before discarding the supernatant), the bacterium is washed twice with pH7.4 PBS, and then the bacterium deposition is stored in a refrigerator at −80° C.

The drug sensitive experiment is carried out according to BACTEC MGIT-960. Each of the RFP MICs of 9 clinical sensitive strains is bellow 0.5 μg/mL. As for the 5 RFP-resistant strains, three of them have a RFP MIC of 32 μg/mL and the other 2 strains have a RFP MIC of 64 μg/mL, respectively.

### EXAMPLE 2

### The Extraction of Bacterial Proteins

20 mL of bacterium in logarithmic growth phase is precipitated by centrifugation at 6000 g, 4 for 15 min, and then the bacterium is washed twice with pH7.4 PBS. The bacterium is dissolved with 0.5 mL bacterial ultrasound buffer, and then the lysate is dissociated with ultrasound in an ice bath for 15 min with a swing 70%, 200 W, ultrasounded every 1 min with a 20 second interval. The sample lysis solvent is added slowly and placed at room temperature for 30 min with intervallic shake, centrifugated at 10 000 rpm, 20 for 15 min, then the supernatant is collected and frozen stored at −80. The protein concentration is determined by Bradford method.

### EXAMPLE 3

### The Extraction of Basic Bacterial Proteins

Twenty milliliters bacterium in logarithmic growth phase is precipitated by centrifugation at 6000 g, 4 for 15 min, and then the bacterium is washed twice with pH7.4 PBS. The bacterium is dissolved with 1 mL PBS and transferred into 1.5 mL EP tubes, and then is centrifugated at 6000 r/min, 4 for 15 min to be precipitated. The resultant bacterium is dissolved with 0.5 mL bacterial ultrasound buffer, and then the lysate is dissociated with ultrasound in an ice bath for 15 min with a swing of 70%, 200 W, ultrasounded every 1 min with a 20-second interval. The bacterium lysate is fixed with pre-cooled methanol at 4 for 40 min (change methanol once after 20 min), and ¼ volume of pre-cooled 0.5 mol/L H₂SO₄ is added slowly. The mixture is stirred for 30 min, then the resultant suspension is centrifugated at 12 000 g, 4 for 20 min. The supernatant is collected and the retained deposition is broken and resuspended in pre-cooled 0.5 mol/L H₂SO₄ for a secondary extraction, centrifugated, and the supernatants are combined together. Quadruple volume cool ethanol is added into the supernatant, stored at −10 for 24 h to precipitate the basic protein. The deposition is collected by centrifugation at 2 000 g for 30 min, and the ethanol is removed as much as possible. Then, the deposition is resuspended in cool ethanol, and centrifugated at 10 000 g for 15 min to collect the deposition. The resultant collection is lyophilized and weighted to subject the analyses detailed as below.

### EXAMPLE 4

### The First Dimension—Immobilized pH Gradients Iso-Electric Focusing Gel Electrophoresis (Electrophoresis Loading Buffer is Made by the Inventors, the Gel Piece is 17 cm, pH4˜7)

The methods of passive hydration and low-voltage in-gel rehydration are used. The hydration loading buffer of −20 is taken from a refrigerator and then dissolved at room temperature. To a tube of the buffer, 0.01 g DTT, 2.5 μl Bio-Lyte 4-6 and 2.5 μl Bio-Lyte 5-7 are added, and the mixture is fully stirred. An IPG prepared gel piece (17 cm, pH 4-7) of −20 is taken from a refrigerator and then placed at room temperature for 10 min. The sample is added linearly along the brim of slot on the focusing plate. After all the samples are added, the protected membrane of the gel piece is removed with a forcep. The IPG piece is set in the sample solution in the focusing plate or hydration plate with the face downward to make sure that the anode (with a “+” marker) of the gel piece corresponds to the anode of the focusing plate. To make sure that the gel piece contacts firmly with the electrode. The plate is stationarily placed at room temperature for 30 min, and then each piece is covered slowly with 2˜3 mL mineral oil, and the anode and cathode are checked again, then a lid is covered. Then the iso-electric focusing program is setup. The limiting current of each piece is 50-70 μA and the focusing temperature is 17.

| hydration | 50 V | 12-16 h (17) | | active hydration |
|---|---|---|---|---|
| S1 | 250 V | linear | 30 min | desalting |
| S2 | 1000 V | fast | 1 h | desalting |
| S3 | 10000 V | linear | 5 h | boost |
| S4 | 10000 V | fast | 60,000 volt hour | focus |
| S5 | 500 V | fast | random | preserve |

After focusing, balance and the second-dimension electrophoresis are carried out immediately, or the gel piece is placed into the sample hydration plate and stored in a −20 refrigerator.

### EXAMPLE 5

### The First Dimension Iso-Electric Focusing Electrophoresis of Basic Protein (“Cup-Loading”, Electrophoresis Loading Buffer is Self-Regulating, the Gel Piece is 17 cm, pH7˜10)

The method of active hydration at 20 is used. The hydration loading buffer is dissolved at room temperature. To a tube of the buffer, 0.01 g DTT, 2.5 μl Bio-Lyte (pH7˜10) are added, and the mixture is fully stirred. Then 330/340 μL buffer is uniformly added into the wells of the hydration plate, placed the IPG piece in the plate with the face downward for 30 min, and then covered with 5 mL mineral oil. The hydration maintains at room temperature for 12˜20 h. The slot of loading piece is cleaned, and the marker is set upon the IEF electrode plate. The hydrated piece is nipped out from the hydration plate, and the mineral oil is decanted, or absorbed with a filter paper wetted by MilliQ water. The piece is set into the piece slot with the face upward, and the cathode of the piece is set to be as close as possible to the (−) end of the slot. A paper salt bridge is set at the connected site of the electrode and the piece surface, and a mobile electrode is set. The surface of the piece is covered with 5 mL mineral oil, and the sample cup is placed outside the mobile electrode at anode end. Then 60 μg (100˜150 μL) sample is added.

The plate is covered with a lid, and the iso-electric focusing program is setup. The limiting current of each piece is 50-70 μA and the focusing temperature is 20.

| | | | | |
|---|---|---|---|---|
| S1 | 250 V | fast | 30 min | desalting |
| S2 | 500 V | fast | 45 min | desalting |
| S3 | 1000 V | fast | 1 h | desalting |
| S4 | 4000 V | linear | 3 h | boost |
| S5 | 4000 V | slow | 60000 volt hour | focus |
| S6 | 500 V | fast | random | preserve |

After focusing, gel balance and the second-dimension electrophoresis are carried out immediately, or the gel piece is placed into the sample hydration plate and stored in a −20 refrigerator.

### EXAMPLE 6

### The Second Dimension SDS-PAGE Electrophoresis

Two pieces of 12% polyacrylamide gel are prepared. The gel is covered with MilliQ water, ethanol or water-saturated butanol so as to keep it flat. The MilliQ water, ethanol or water-saturated butanol is removed after gelatinization, and the gel is washed with MilliQ water. Then a gel piece is taken from −20 refrigerator and placed at room temperature for 10 min to dissolve. Upon gelatinization, the excessive water, mineral oil and excessive sample on the piece is absorbed with filter paper wetted by MilliQ water. Then the piece is transferred into a swelling disk, and 5 mL piece balance buffer I is added. The disk is shaken slowly in a shaker for 12 min. Then, the entire piece balance buffer I in the sample hydration disk is removed or absorbed thoroughly, and the piece balance buffer II is added. And the disk continues to be shaken gently in the shaker for 14 min. Then, the IPG piece is removed from sample hydration disk, and one end of the piece is nipped with a forcep and is soaked into 1× electrophoresis buffer. The piece is then placed on a long glass plate with the surface of gel piece upward. Each piece is operated in the same way. Low melting-point agarose sealing liquid is added upon the gel, and the piece contacts fully with a polyacrylamide gel. The 2-DE electrophoresis is performed after the sealing fluid coagulates. At the beginning low-current (5 mA/gel/17 cm) or a low-voltage is used, the current or voltage is increased when samples have come out of the IPG piece and concentrated to a string. When the bromophenol blue indicator has reached the edge of the bottom, stop the electrophoresis. The gel is taken out and cut a corner as a marker.

### EXAMPLE 7

### The Silver-Staining of Protein

After the electrophoresis, the gel is taken out and immersed in the fixing solution for a 30 min treatment. After the fixing solution is removed, the dipping solution is added for a further 30 min treatment. Then the gel is washed for 5 min with MilliQ water three times. Then, the silver-staining solution is added for a 20 min staining, thereafter the silver-staining solution is removed, and the coloration solution is added. The shaking of the shaker is accelerated. Upon the coloration is visibly finished, the stop solution is added to stop the coloration. The whole coloration process is carried out on a shaker plate at room temperature.

### EXAMPLE 8

### Analysis of Gel Photograms

A gel is scanned by a Molecular Image Fx Laser Imaging Scanner. The differential expression sites of proteins are screened by the comparison among the bacterial proteins gel photogram of a clinical drug-resistance M.tb, that of a clinical sensitive M.tb and that of the standard strain H37Rv, the comparative analysis is carried out by the software PDQuest 6.0.

### EXAMPLE 9

### Analysis of MALDI-TOF-MS Peptide Mass Fingerprinting

The protein blots of the differential expression among the bacterial proteome from the clinical drug-resistance M.tb, that from the clinical sensitive M.tb and that from the standard strain H37Rv are selected respectively, and then cut down and put into sample tubes. The dig protein blots are then cut into chips respectively and decolored with 50% acetonitrile. Then 100% acetonitrile is added to make the gel shrink and white. After the supernatant is removed, an appropriate amount of 0.1 mol/L NH₄HCO₃ is added. The same volume of acetonitrile is added after 5 min, then the gel chip is vacuum-dried after 15 min. An appropriate amount of 10 mmol/L DTT/0.1 mol/L NH₄HCO₃ is added and incubated in a water bath at 56 for 45 min. Then the supernatant is removed, and the same volume of 55 mmol/Liodoacetamide/0.1 mol/L NH₄HCO₃ is added, lucifugously incubated at room temperature for 30 min. An appropriate amount of 0.1 mol/L NH₄HCO₃ is added, and then the same volume of acetonitrile is added after 5 min. After 15 min, the gel is vacuum-dried, and an appropriate amount of digest (50 mmol/L NH₄HCO₃, 5 mmol/L CaCl₂, 12.5 ng/μl Trypsin) is added. After 30 min at 4, the remained liquid is sucked out, and an appropriate amount of enzyme-free digest is added and incubated at 37 overnight. The solvent 25 mmol/L NH₄HCO₃ is added, and the same volume of acetonitrile is also added after 15 min, and then an appropriate amount of 5% TFA is added after 15 min and incubated at 40 for 1 h. The supernatant is collected, and 2.5% TFA/50% acetonitrile is added and incubated at 30 for 1 h. Then the supernatants are combined together and vacuum-dried, and dissolved with 0.5% TFA. Then the MALDI-TOF-MS peptide mass fingerprinting analysis is carried out.

### EXAMPLE 10

### Searching the Database

The matching of peptide mass fingerprinting is achieved by PepIdent (www.expasy.org/tools/peptident.pl). Parameters are set as: species—bacterium, Mw—apparent molecular weight ±30%, pI—random; enzyme—pancreatin, enzymatic cleavage sites could be ignored—1, modification type—iodacetyl. The matching of amino acid sequence domain is achieved by Prosite Scan (www.expasy.org/tools/prosite scan.pl). The homology search of amino acid is achieved by Blast (www.ncbi.nlm.nih.gov/blast/Blast.cgi).

Finally, the present invention has disclosed 13 proteins as shown in table 1, and some of them are already known and have demonstrated to be closely related to M.tb.

TABLE 1

| | | | | | | | pI | | Expression |
|---|---|---|---|---|---|---|---|---|---|
| No. | protein | gi in NCBI | Locus tag | PMF score | MS/MS | | theoretical/experimental | MW theoretical/experimental | In clinical strains |
| R1 | Ssb | gi\|15607196 | Rv0054 | 234(5/6) | Parent ion: 1655.885MH+ | | 4.84/5.12 | 17320.99/ | decrease |
| | | | | | Peptide: TVIEVEVDEIGPSLR | Score: 43 | | 17342.6 | |
| | | | | | Parent ion: 1793.918MH+ | | | | |
| | | | | | Peptide: TPSGAAVANFTVASTPR | Score: 89 | | | |
| | | | | | Parent ion: 1957.0236MH+ | | | | |
| | | | | | Peptide: AGDTTITIVGNLTADPELR | Score: 55 | | | |
| R2 | Hypothetical protein | gi\|15607210 | Rv0068 | 58(5/11) | Parent ion: 1499.6913MH+ | | 8.82/8.07 | 32398.79/ | disappear |
| | | | | | Peptide: GGQYFGPDGFGEIR | Score: 11 | | 32379 | |
| R3 | Hypothetical protein | gi\|15608067 | Rv0927 | 132(6/11) | Parent ion: 1418.7122MH+ | | 5.6/5.49 | 26745.62/ | disappear |
| | | | | | Peptide: TSSELDAVAEQIR | Score: 71 | | 26729.1 | |
| | | | | | Parent ion: 2056.1548MH+ | | | | |
| | | | | | Peptide: GASLRGAAIALAFAQAGADVLI | Score: 16 | | | |
| R4 | BfrB | gi\|15610977 | Rv3841 | 112(6/10) | Parent ion: 1228.6531MH+ | | 4.47/4.73 | 20441.93/ | increase |
| | | | | | Peptide: EALALALDQER | Score: 21 | | 20429.2 | |
| | | | | | Parent ion: 1550.796MH+ | | | | |
| | | | | | Peptide: AGANLFELENFVAR | Score: 49 | | | |
| R5 | OpcA | gi\|15608584 | Rv1446 | 143(6/18) | Parent ion: 1212.731MH+ | | 4.98/5.2 | 32717.49/ | decrease |
| | | | | | Peptide: AVGELKVELVR | Score: 29 | | 32697.4 | |
| | | | | | Parent ion: 1360.7218MH+ | | | | |
| | | | | | Peptide: VGADAGAGEFVVLR | Score: 38 | | | |
| | | | | | Parent ion: 1237.7262MH+ | | | | |
| | | | | | Peptide: TGKPDALVPLAR | Score: 15 | | | |
| R6 | Antigen 84 | AG84_MYCTU | Rv2145 | 133(6/16) | Parent ion: 1565.7805MH+ | | 4.52/4.8 | 28277.1/ | increase |
| | | | | | Peptide: TYLESQLEELGQR | Score: 10 | | 28260.1 | |
| | | | | | Parent ion: 1995.879MH+ | | | | |
| | | | | | Peptide: GFNSRAAPVDSNADAGGFDQ | Score: 81 | | | |
| 7# | Hypothetical protein | gi\|15609296 | Rv2159 | 189(5/13) | Parent ion: 1515.7914MH+ | | 7.82/7.21 | 36376.38/ | decrease |
| | | | | | Peptide: IGTWIGAAAEGQVSR | Score: 12 | | 36353.9 | |
| | | | | | Parent ion: 2147.1455MH+ | | | | |
| | | | | | Peptide: LAAALRLTGLAPHQVTDDDVA | Score: 70 | | | |
| R8 | CysK | gi\|15609471 | Rv2334 | 91(6/12) | Parent ion: 1356.7885 MH+ | | 4.93/5.2 | 32752.62/ | increase |
| | | | | | Peptide: LIVVVLPDFGER | Score: 32 | | 32732.3 | |
| | | | | | Parent ion: 2028.0085MH+ | | | | |
| | | | | | Peptide: YFVPQQFENPANPAIHR | Score: 17 | | | |
| R9 | Hypothetical protein | gi\|15609766 | Rv2629 | 187(4/15) | Parent ion: 1194.7205MH+ | | 5.01/5.2 | 40839.76/ | new |
| | | | | | Peptide: IAPLDGVGALLR | Score: 14 | | 40900 | |
| | | | | | Parent ion: 1801.933MH+ | | | | |
| | | | | | Peptide: LVDAADPEVVFVSGEVR | Score: 10 | | | |
| 10# | ilvN | gi\|15610139 | Rv3002 | 70(2/3) | Parent ion: 1275.7054MH+ | | 8.52/7.93 | 18186.89/ | new |
| | | | | | Peptide: SQVIEAVNLFR | Score: 38 | | 18175.8 | |
| R11 | DevR | gi\|15610269 | Rv3133c | 90 | Parent ion: 1477.8485MH+ | | 5.45/5.62 | 23261.87/ | increase |
| | | | | | Peptide: VPAARPDVAVLDVR | Score: 21 | | 23279.3 | |
| R12 | PPE | gi\|15610272 | Rv3136 | 73(6/7) | Parent ion: 1559.7886MH+ | | 4.11/4.37 | 37979.75/ | decrease |
| | | | | | | | | 37956.2 | |
| 13# | nuoB | gi\|15610282 | Rv3146 | 55(4) | Peptide: MDFALLPPEVNSAR | Score: 62 | 7.95/7.68 | 20186.78/ | disappear |
| | | | | | | | | 20433.5 | |

The 13 proteins shown in table 1 are classified according to their function, and the classification criterions are shown at the website (http://genolist.pasteur.fr/TubercuList/). Class 0: virulence, detoxifcation, acclimatization, Class 1: lipoid compound metabolization, Class 2: signal pathway, Class 3: cell wall and cell process, Class 5: inserted sequece and antibiotic, Class 6: PE/PPE, Class 7: intermediates of metabolization and respiration, Class 8: unknown, Class 9: regulation protein, Class 10: conservative hypothetical protein.

The functions of most RFP-resistant proteins are classified to class 7 and class 10, the two proteins have functions of an intermediate of metabolism or respiration and a conservative hypothetical protein, especially the former. It can be concluded that after the bacteria possess the RFP-resistance, some proteins participating in the metabolism and respiration will vary so as to affect the RFP-resistance by regulating the metabolism.

R1, which is Ssb, or Rv0054, belongs to the signal protein which plays an important roles in DNA metabolism (include gene copy, repair and recombination) [Saikrishnan K., Jeyakanthan J., Venkatesh J., Acharya N., Sekar K., Varshney U., Vijayanl M. Structure of Mycobacterium tuberculosis Singlestranded DNA-binding Protein. Variability in Quaternary Structure and Its Implications[J]. J. Mol. Biol., 2003,331:385-393; Mothe Sreedhar Reddy, Guhan N., Muniyappa K. Characterization of Single-stranded DNA-binding Proteins from Mycobacteria[J]. The Journal of Biological Chemistr, 2001, 276(49): 45959-45968.]. Protein SsB is linked to a single strand DNA with high affinity, and the absorption constant and desorption constant between them are the same. The combination of them can alter the domain at the carboxyl end of the protein SsB which is the binding site of SsB and its homological RecA [Ajay Kumar R., Moreshwar B. Vaze, Nagasuma R. Chandra, Vijayan M., Muniyappa K. Functional Characterization of the Precursor and Spliced Forms of RecA Protein of Mycobacterium tuberculosis[J]. Biochemistry, 1996, 35:1793-1802, Priya Handa, Narottam Acharya, Umesh Varshney. Chimeras Between Single-stranded DNA-binding Proteins from Escherichia coli and Mycobacterium tuberculosis Reveal That Their C-terminal Domains Interact with Uracil DNA Glycosylases[J]. The Journal of Biological Chemistry, 2001, 276 (20): 16992-16997]. SsB and RecA are two kinds of proteins with high affinity and they often act as regulating roles together. SsB exhibits high conservation in Mycobacterium strains, and its characteristic conformation includes a lot of hairpin structures in the secondary structure and the form of elliptical tetramer. This functional tetramer is different from that in E. coli [Hans Joachim Mollenkopf, Leander Grode, Jens Mattow, Maik Stein, Peggy Mann, Bernhard Knapp, Jeffrey Ulmer, Stefan H. E. Kaufmannl. Application of Mycobacterial Proteomics to Vaccine Design: Improved Protection by Mycobacterium bovis BCG Prime-Rv3407 DNA Boost Vaccination Against Tuberculosis [J]. Infection and Immunity, 2004, 72(11): 6471-6479.], and the form binding to DNA is also different from that in E. coli. Therefore, SsB can be taken as a molecular target to control the M.tb's growth. It is also found that SsB is down-regulated expressed both in a drug-resistance strain and in a clinical sensitive strain, which indicate a common variation of the clinical strains in the infection, which brings the difference between the clinical strains and experimental standard sensitive strains.

R2, which is Rv0068, is a kind of hypothetical protein or a kind of oxidoreductase, which acts in the respiration pathway. Hans, etc. have study the bacterial proteins from M.tb and M. bovis by proteomic analysis and found that Rv0068 is only expressed in M.tb but not in M. bovis [Betts J. C., Dodson P., Quan S., Lewis A. P., Thomas P. J., et al. Comparison of the Proteome of Mycobacterium tuberculosis Strain H37Rv with Clinical Isolate CDC 1551[J]. Microbiology, 2000, 146:3205-3216]. The result indicates that Rv0068 may be used for the serological detection or used as a drug target of M.tb. It is found in the invention that Rv0068 is not expressed in single-drug-resistant M.tb and down-regulation expressed in a multi-drug-resistance strain. The result indicates that the respiration of a drug-resistance strain reduces, which may be related to the activity of a clinical drug-resistance strain.

R3, which is Rv0927c, is also a hypothetical protein, and their functions relate to dehydrogenase or reductase. Betts etc. has studied the proteins expressed by M.tb H37Rv and CDC1551 in different growth phases by means of proteomic analysis and found that Rv0927c is specifically expressed in CDC1551 but not expressed in H37Rv with the prolong of the culture time [7]. In the present invention, we found that Rv0927c is not expressed in clinical RFP-resistant strains and clinical sensitive strains but is highly expressed in an H37Rv strain which is used as a control. The difference of the two above results may be related to the cell culture time. The bacteria tested in the experiment are in logarithmic growth phase, whereas Betts described the protein expression in the bacteria growing until the end of stationary phase. Since the synthesis of cellular protein is in schedule, the protein expression is different in the growth time and growth state. In addition, the strains used in the two experiments are different, and the strains used in our experiment belong to H37Rv Beijing-family-type, which is epidemic in Asia but seldom appears in Europe and America. Though the protein expression analysis of different infectious M.tb families has not been reported in detail heretofore, different protein expression maybe exists in M.tb strains of different sources and epidemic modes.

R4, which is BfrB or Rv3841, is a kind of bacterial ferritin, which is a subunit of a macromolecular membrane protein with high immunogenicity [Brooks B. W., Young N. M., Watson D. C., Robertson R. H., Sugden E. A., Nielsen K. H., Becker Sawe. Mycobacterium-Paratuberculosis Antigen-D-Characterization and Evidence That It Is a Bacterio ferritin[J]. Journal of Clinical Microbiology, 1991, 29 (8): 1652-1658]. The amino acid sequence analysis indicates that the motif sequence of a ferroxidase center in bacterial ferritins is highly conservative, and the gene sequence encoding motif of the ferroxidase center is of high sequence homology in Mycobacterium paratuberculosis, Mycobacterium avium, Mycobacterium tuberculosis, Mycobacterium scrofulaceum. The result indicates that each of BfrB, Rv3841 and Rv3841 is an essential protein of M.tb strains [Pessosani M C V., Smith D. R., Rivoire B., Mccormick J., Hefta S. A., Cole S. T., Brennan P. J. Purification, Characterization, Gene Sequence, and Significance of a Bacterioferritin from Mycobacterium leprae[J]. Journal of Experimental Medicine, 1994, 180(1): 319-327]. In terms of the functions, ferritin is closely related to the bacterial nitrogen metabolism including the nitrogen oxidation and the NO reduction. The actions of nitric oxide on the determination of M.tb infection to the host has always been an interest. It is reported that the protein expression changes in the strain H37Rv and strain 12646 after the strains are treated with nitric oxide, including the BfrB up-regulation. If the phenomena are combined with the immunoreaction detection between BfrB and the M.tb infected subjects' and the TB patients' serums, the result will indicate the effect of BfrB on the M.tb infection [Garbe T. R., Hibler N. S., Deretic V. Response to Reactive Nitrogen Intermediates in Mycobacterium tuberculosis: Induction of the 16-kilodalton a-crystallin Homolog by Exposure to Nitric Oxide Donors [J]. Infect. Immun, 1999, 67:460-65]. So far, the study on BfrB is limited and the process is not searched in the database. BfrB is significantly up-regulated expressed in the RFP-resistant strain and is slightly up-regulated expressed in the sensitive strain. The result shows that BfrB may be influenced by the degree of drug resistance, and special attention must be paid to the RFP-resistant mechanism of M.tb and the relationship between nitric oxide and M.tb infection.

R5, which is OpcA encoded by the gene Rv1446c, is an outer membrane protein exposed on the cell surface with 10 transmenbrane side-chains and 5 outer membrane loops. The transmenbrane domains of OpcA are of high conservation, while the outer membrane loops and the turn formations in periplasm have diversity among microorganisms. In many pathogenic bacteria, OpcA mediates the adhesion of bacteria to endothelial cells and epithelial cells, the cell invasion and the inner cellular localization. OpcA mainly interacts with the integrin of endothelial cells [Moore Jeremy, Bailey Simon E. S., Benmechernene Zineb, Tzitzilonis Christos, Griffiths Natalie J. E., Virji Mumtaz, Derrick Jeremy P. Recognition of Saccharides by the OpcA, OpaD, and OpaB Outer Membrane Proteins from Neisseria meningitidis[J]. Journal of Biological Chemistry, 2005, 280(36): 31489-31497; Peixuan Zhu, Giovanna Morelli, Mark Achtman. The opcA and opcB Regions in Neisseria: Genes, Pseudogenes, Deletions, Insertion Elements and DNA Islands[J]. Molecular Microbiology, 1999, 33(3):635-650]. OpcA expression quantity is highly variable due to different transcription level, which mainly depends on the length of polycytosine side-chain at the gene initiation region. Though the protein has been expressed in M.tb, further corresponding information has not as yet been reported. The result shows that the expression of OpcA is down-regulate in a drug-resistant strain and in a sensitive strain, especially in the former.

R6, which is Wag31 or Rv2145c, is a highly conservative protein in M.tb. The 2-DE electrophoresis proves that each of them locates at the outer membrane or the wall [Peter R. Jungblut. Proteome Analysis of Bacterial Pathogens[J]. Microbes and Infection, 2001, 3:831-840], and it is a kind of secretory surface glyco/lipopolysaccharide protein with immunogenicity [Andrew E. Greenstein, Christoph Grundner, Nathaniel Echols, Laurie M., Gay T., Noelle Lombana, Carl A. Miecskowski, Kristi E. Pullen, Pei-yi Sung Tom. Structure/Function Studies of Ser/Thr/Tyr Protein Phosphorylation in Mycobacterium tuberculosis[J]. J Mol Microbiol Biotechnol, 2005, 9:167-181]. In vivo, Wag31 is closely related to the function of Ser/Thr/Tyr protein kinase (Pkn). The coaction of PknA and PknB could result in the intense phosphorylation at 73-Thr of Wag31, so as to regulate the cell division and the cell morphology [Andrew E. Greenstein, Christoph Grundner, Nathaniel Echols, Laurie M., Gay T., Noelle Lombana, Carl A. Miecskowski, Kristi E. Pullen, Pei-yi Sung Tom. Structure/Function Studies of Ser/Thr/Tyr Protein Phosphorylation in Mycobacterium tuberculosis[J]. J Mol Microbiol Biotechnol, 2005, 9:167-181]. Wag31 has 260 amino acid residues. It is found by the analysis of MS-MS that 8 amino acid residues of Wag31 in M.tb have been phosphorylation modified, which locate at Thr, Ser and a Tyr residue respectively.

The response in a metabolic level to the internal and external environment and the self-specific physiological state is an essential feature of life. Reversible protein phosphorylation is a sensitive physiological signal, by which the organism can correspondingly respond to different effect factors including cell type, virulence, chemotaxis, pathogenicity and so on. Compared with that in the strain H37Rv, Wag31 is rarely expressed in a clinical sensitive strain, but is up-regulated in a RFP-resistant strain. The multi-site phosphorylation of Wag31 indicates that Wag31 is closely related to the RFP-resistance. It needs to be further studied to prove whether the multi-site phosphorylation is a reason of RFP-resistance or the result.

7#, which is Rv2159, is a conservative hypothetical protein [Lia Danelishvili, Martin Wu, Lowell S. Young, and Luiz E. Bermudezl. Genomic Approach to Identifying the Putative Target of and Mechanisms of Resistance to Mefloquine in Mycobacteria[J]. Antimicrobial Agents and Chemotherapy, 2005, 40(9): 3707-3714]. It is proved that either of them belongs to a membrane protein of M.tb by the proteomic analysis [Colette Goffin, Jean-Marie Ghuysen. Biochemistry and Comparative Genomics of SxxK Superfamily Acyltransferases Offer a Clue to the Mycobacterial Paradox: Presence of Penicillin-Susceptible Target Proteins versus Lack of Efficiency of Penicillin as Therapeutic Agent[[J]. Microbiology and Molecular Biology Reviews, 2002, 66(4): 702-738]. Function of Rv2159c is still unknown. The gene encoding Rv2159c has a high sequence homology with the gene ddl (encoding a ligase used during the formation of D-alanyl-D-alanine) in E. coli, and this gene locates at other site of chromosome in M.tb, M. leprae and S. coelicolor [Colette Goffin, Jean-Marie Ghuysen. Biochemistry and Comparative Genomics of SxxK Superfamily Acyltransferases Offer a Clue to the Mycobacterial Paradox: Presence of Penicillin-Susceptible Target Proteins versus Lack of Efficiency of Penicillin as Therapeutic Agent [[J]. Microbiology and Molecular Biology Reviews, 2002, 66(4): 702-738]. Regulation of gene expression appears through the infection process of M.tb in which transcription regulator plays an important role. In M.tb, many δ factors regulate the genes' expressions under the environmental stresses, and said genes include some essential genes to the M.tb virulence or M.tb sustainable infection. Raman has found that gene Rv2159 expression quantity decreased by 1.7 times in a sigD gene-deleted mutant strain, whereas the sigD gene expression quantity was stable in the wild-type H37Rv strain. The later did not change along with the cell life cycle, but it decreased significantly with the oxygen consumption, and the bacterial infectious ability also decreased significantly. The synchronization characteristics of the in vivo expression of the genes Rv2159c and sigD indicates their functions are closely related [Sahadevan Raman, Rohan Hazra, Christopher C. Dascher, and Robert N. Husson. Transcription Regulation by the Mycobacterium tuberculosis Alternative Sigma Factor SigD and Its Role in Virulence[J]. Journal of Bacteriology, 2004, 186(19):6605-6616]. Our study proves a decreasing tendency of R7 protein expression in the drug-resistant strain, which indicates that the appearance of drug resistance may lead to that the bacterial infectious ability to a host declines and the latent infectious ability in vivo is influenced.

R8, which is CysK or Rv2334, its crystal has been obtained and the crystal's structure has been analyzed. There are two steps in the biosynthesis of L-Cys, and the second step is O-acetyl-L-serine-thiolyase catalysis in which pyridoxal phosphate is used as prosthetic group and O-acetyl is replaced by sulfhydryl. One of the two isoenzymes catalyzing the second step is CysK, which takes the sulfide as a substrate so as to synthesize L-Cys [Tobias Daûler, Thomas Maier, Christoph Winterhalter, August BoÈck. Identification of a Major Facilitator Protein from Escherichia coli Involved in Efflux of Metabolites of the Cysteine Pathway[J]. Molecular Microbiology, 2000, 36(5):1101-1112]. Cys anabolism is the main manner for a microorganism to absorb sulfur, and the mutant of gene cysK usually result in the variation of almost all essential genes for sulfate assimilation, therefore, Besides used as the catalyzer of cysteine synthesis, CysK is also a negative gene regulator in sulfur metabolism [Albanesi Daniela, Mansilla Maria Cecilia, Schujman Gustavo E., de Mendoza Diego. Bacillus subtilis Cysteine Synthetase Is a Global Regulator of the Expression of Genes Involved in Sulfur Assimilation[J]. Journal of Bacteriology, 2005, 187(22):7631-7638].

In a study on Moorella thermoacetica, CysK in cytoplasm is inductively expressed by removing the conventional reductant. The result indicates that CysK may be related to the protective mechanism for bacteria to resist against the oxygen stress, which enable the bacteria to endure the aerobic environment [Das Amaresh, Silaghi-Dumitrescu Radu, Ljungdahl Lars G., Kurtz Jr. Donald M. Cytochromebd Oxidase, Oxidative Stress, and Dioxygen Tolerance of the Strictly Anaerobic Bacterium Moorella thermoacetica[J]. Journal of Bacteriology, 2005, 187(6):2020-2029]. But an abnormal expression of CysK is not been found in the comparative proteome analysis in the growth of M.tb under aerobic and anaerobic conditions [Joakim Starck, Gunilla Ka\ddot{} llenius, Britt-Inger Marklund, Dan I. Andersson, Thomas A^{∘}kerlund. Comparative Proteome Analysis of Mycobacterium tuberculosis Grown Under Aerobic and Anaerobic Conditions[J]. Microbiology, 2004, 150:3821-3829]. Up-regulated expression of CysK in all clinical strains indicates that this protein may be unrelated to the M.tb drug resistance. The result demonstrates that metabolisms of M.tb's amino acids especially Cys, Met and Ser change in the clinical strains, and the adaptability of the clinical strains to the high oxygen environment increases, which means the increased adaptability to the living environment and epidemic mode.

R9, which is Rv2629, is a hypothetical protein with unknown function. So far, just only one literature about this protein has been reported [Jack C. Tarleton, Bert Ely. Isolation and Characterization of ilvA, ilvBN, and ilvD Mutants of Caulobacter crescentus[J]. Journal of Bacteriology, 1991, 173(3):1259-1267]. Rv2629 is regarded as a specific protein appearing under environmental stress and a protein marker related to the latent infection due to its up-regulated expression under a low concentration nitric oxide induction or an anaerobic condition. Our experiments indicates that the protein is not expressed in H37Rv and is slightly expressed in a clinical sensitive strain whereas obviously increases in all drug-resistant strains including multi-drug-resistant strains, which is similar to that in the anaerobic condition. It can be deduced that a bacterium reduces its living ability and enters into a latent state so as to resist against the disbenefit external environment, or a bacterium reduces the drug's efficiency by entering into a latent state so as to escape from sterilizing effect of antibiotics.

10#, which is ilvN or Rv3002c, is a new point observed by basic protein gel detection. The result that it exists in all drug-resistant strains indicates a close relationship between the protein and the drug resistance of M.tb. ilvN belongs to branched amino acid family. Acetohydroxyacid synthases (AHAS) is catalyzer for the synthesis of isoleucine and valine. There are two kinds of AHAS isozymes in M.tb, which are AHAS I and AHAS II. The former comprises a big subunit ilvB (Rv3003c), ilvB2 (Rv3470c) and a small subunit ilvN (Rv3002c), while the later is a enzyme comprises a single subunit ilvG (Rv1820), which shows a great different structure from most of other prokaryotes [Lillian Eoyang, Philip M. Silverman. Role of Small Subunit (IlvN Polypeptide) of Acetohydroxyacid Synthase I from Escherichia coli K-12 in Sensitivity of the Enzyme to Valine Inhibition[J]. Journal of Bacteriology, 1986, 166(3):901-904]. IlvN determines the sensitivity of AHAS I to the feed-back control of valine [Sharmila Anishetty, Mrudula Pulimi, Gautam Pennathur. Potential drug targets in Mycobacterium tuberculosis through metabolic pathway analysis[J]. Computational Biology and Chemistry, 2005, 8:1-12]. Anishetty analyzed the metabolic pathway of human and M.tb by computer, the enzymes of the biochemical metabolisms of M.tb in KEGG metabolic database are compared with its host that human's non-redundant proteins, so as to screen the potential drug targets for further research. 185 potential drug targets are found and 86 of them exist in amino acid metabolic pathway. IlvN (Rv3002c), ilvC (Rv3001c), ilvD (Rv018c), isopropyl malate synthase leuA (Rv3710) and the small subunit leuD (Rv2987c) of isopropyl malate dehydrogenase may be taken as potential drug targets for the isolation of antituberculotic in the Val/Leu/Ile metabolic pathway [Sharmila Anishetty, Mrudula Pulimi, Gautam Pennathur. Potential drug targets in Mycobacterium tuberculosis through metabolic pathway analysis[J]. Computational Biology and Chemistry, 2005, 8:1-12].The new increased expression of IlvN in clinical drug-resistant strains is detected by proteomic analysis, which can be taken as an effective mode to design a drug target for M.tb, especially for drug-resistant M.tb, and taken as a standard for drug-resistance detection.

R11, which is DevR or Rv3136, is a transcription regulator in the bicomponent regulator family, and it is the earliest mediator in M.tb signal in the hypoxia condition [Heui Dong Park, Kristi M. Guinn, Maria I. Harrell, Reiling Liao, Martin I. Voskuil, Martin Tompa, Gary K. Schoolnik, David R. Sherman. Rv3133c/dosR is a Transcription Factor That Mediates the Hypoxic Response of Mycobacterium tuberculosis[J]. Molecular Microbiology, 2003, 48 (3):833-843]. M.tb's latent state in its host is related to the hypoxia condition. Induction of DevR is essential to almost all of hypoxia response gene which locates in the downstream region of gene Rv3133c encoding DevR [David R. Sherman, Martin Voskuil, Dirk Schnappinger, Reiling Liao, Maria I. Harrell, Gary K. Schoolnik. Regulation of the Mycobacterium tuberculosis Hypoxic Response Gene Encoding a-crystallin[J]. PNAS, 2001, 98(13): 7534-7539]. The combination of Rv3133/DosR with a small heat shock protein α-crystal glubin (Acr) completes the regulation. The encoding gene of Acr locates in the up-stream region of hypoxia response gene. The inducible expression of Acr is induced by DevR under reduction conditions, and the down-regulated expression of Acr is reduced by DevR in a hypoxia condition with carbon dioxide. DevR may be a general regulator for the promoters of the most ACG family members. Thus, Rv3133 is essential for M.tb to be latently growth under the hypoxia condition [Matthew A. Florczyk, Lee Ann McCue, Anjan Purkayastha, Egidio Currenti, Meyer J. Wolin, Kathleen A. Mcdonough. A Family of acr-Coregulated Mycobacterium tuberculosis Genes Shares a Common DNA Motif and Requires Rv3133c (dosR or devR) for Expression[J]. Infection and Immunity, 2003, 71(9): 5332-5343].

M.tb infection in vivo will lead to the appearance of granuloma. In the granulation, M.tb enters a stationary resistant state without gene replication, but few corresponding details have been reported. It has been a focus for people to regard the hypoxia state in granulation as the signals of M.tb latent and persistent infection. Rv3133 is crucial in this process.

DevR is up-regulation expressed in all clinical drug-resistant strains, whereas its expression is changeless in sensitive strains. Here, a lot of DevR will induce the down-regulation expression of Acre, which will promote the bacteria to enter into the resistant state without gene replication.

M.tb latent infection is always a serious public health problem. Presently, all antituberculotic are designed against the bacteria in active state, thus the existence of latent M.tb make the traditional TB treatment more difficult. The discovery of Rv3133 offers a new approach for us to control the latent and drug-resistant M.tb.

The current experiments show that devR exists in all M.tb samples and clinical isolates, and devR differential expression exists in M.tb strains. In clinic, the PCR amplification of 250-500 bp fragments from the DevR (Rv3133c) gene significantly increases the sensitivity and efficiency of Tuberculosis diagnosis [Soumitesh Chakravorty, Divya Pathak, Mridu Dudeja, Sagarika Haldar, M. Hanif, Jaya Sivaswami Tyagi. PCR Amplification of Shorter Fragments From the DevR (Rv3133c) Gene Significantly Increases the Sensitivity of Tuberculosis Diagnosis[J]. FEMS Microbiol Lett, 2006, 257:306-311]. Our result of proteomic analysis has also proved the works of forerunner well.

R12 is PPE. It should be note that Rv3136c, which is a PPE family member, has been isolated in our experiments. No information of its function has ever been reported though the in vivo expression of PE and PPE family member has been reported before [Singh K. K., Zhang X., Patibandia A. S., Chien P. J. R., Laal S., Antigens of Mycobacterium tuberculosis Expressed During Preclinical Tnb-erculosis: Serological Immunodominance of P-roteins with Repetitive Amino Acid Sequence[J]. Infection and Immunity, 2001, 69(6):4185-4191; Triccas J. A., Berthet F. X., Pelicic V., et al. Use of Fluorescence Induction and Sucrose Counterselection to Identify Mycobacterium tuberculosis Genes Expressed Within Host Cells[J]. Microbiology-UK, 1999, 145: 2923-2930; Li Ting, Gao Zhiyong, Wang Hengdong, et al. M.tb Expression Screening by Antibody Induction Technique[J]. Genetics Transaction 2005, 2 (2):111-117.]. The PPE protein family is regarded as a “molecular curse” of M.tb because it is a huge protein family with members as many as 100 and 67, which occupy 8% of M.tb genome. But the function of this family is still unknown. The present study initially indicates that some of PE and PPE proteins may be cell surface antigens related to M.tb virulence and pathogenicity. Moreover, the molecular and physiochemical analysis of single gene is very difficult due to the high sequence homology of PE/PPE family [Sailu Yellaboina, Jayashree Seshadril, M. Senthil Kumar, Akash Ranjan. Predict Regulon: a Web Server for the Prediction of the Regulatory Protein Binding Sites and Operons in Prokaryote Genomes[J]. Nucleic Acids Research, 2004, 32:318-320]. The expression of this protein is widely down-regulated or absent in clinical strains in our study. The reasons lie in that Rv3136 is down-regulation expressed in clinical strains, but it is more impossible that the protein is secreted into the extracellular matrix and acts as an antigen to enhance pathogenicity of clinical strains.

13#, which is nuoB or Rv3146, belongs to a NADH-dehydrogenase B chain which is called as NADH-COQ oxidoreductase B chain. The crystal structure of the protein has been disclosed. As subunit of respiration catalyzer, the function of nuoB is specific such that corresponding study on it is seldom reported. In recent years, only one paper referred that nuoB may be the binding site of LexA which is a M.tb transcription regulator. But it is just a comparative primer designed according to the KexA gene sequence of B. subtili, and the comparative result from the database needs to be further certified by experiments. Basic protein analysis of drug-resistant strains shows the expression of nuoB is of big fluctuation but asynchronous. nuoB is absent in RFP-resistant strains, whereas it is up-regulation expressed in a multi-drug-resistant strain. The result indicates that M.tb drug resistance is related to the changes of its respiration, but different drug-resistant mode brings different influence to its respiration, and it is suggested that different drug-resistant strains have specific drug-resistance mechanisms.

After summarizing the functions of all the 13 proteins related to the RFP-resistance, it is found that the functions of 3 proteins (Rv2159, Rv2629 and DevR) have direct relationship with the M.tb latent and persistent infection. Each mode of the changes in drug-resistant strains leads to that its infection ability declines, its living ability decreases and the strain enters into a latent state. M.tb decreases its interaction efficiency with the drug in such a manner so as to resist against the sterilizing effect of antibiotics and to enhance its drug resistance. The result implies that the infection ability and the living ability of clinical drug-resistant strains decrease, but the strain escapes from drug's effect by entering into a latent state. The combination with other functions of the bacterial protein ensures that M.tb has some biological activity, drug resistance and infectivity.

In example 5, all experiments are carried out in conventional methods. The genome extraction is carried out according to the protocol of Shanghai Huashun Company, except that the digestion time is prolonged from 0.5 h to 4 h at 37 water bath treating with Lysozyme and RNaseA, so as to ensure the absolute lysis of the extra-thick cell wall of M.tb.

PCR amplification of specific gene is performed by using a gene sequence obtained from GENEBANK and the primers designed by OLIGO 6.0 software. The target protein band is isolated by 1% agar-gel electrophoresis of PCR product and purified by QIA quick column PCR purification kit (QIAGEN). And then, the protein is subject to sequence analysis in a ABI 377 (Applied Biosystems, Inc.) automatic sequence analyzer by Shanghai Sangon Biotech Company or Shanghai Branch of Beijing AuGCT Biotech Company. The resultant sequence is compared with the corresponding sequence of H37Rv standard strain, and the result is shown in Table 2. The A/C mutant at nucleotide 191 of gene Rv2629 is shown in FIG. 13.

The comparison and analysis show that A/C mutant at nucleotide 191 of gene Rv2629 is closely related to the M.tb RFP-resistance. Accordingly we increase the collection of strains and the gene sequencing analysis. While the sequences of gene rpoB and Rv2629, A/C mutant at nucleotide 191 of gene Rv2629 are analyzed, and it is found that the A/C mutant at nucleotide 191 of gene Rv2629 appears in 111 strains among the 112 RFP-resistant strains, with a high mutation rate of about 99.11%; and the mutation rate of rpoB gene is 90.18% (101/112), and specific mutation rate of different sites are shown in table 3. The A/C mutant at nucleotide 191 of gene Rv2629 is not found in 30 clinical RFP-sensitive strains.

In example 6, the PCR primers are designed according to the gene structure of Rv2629 and the polycloning site characteristics of pET30 and pMV261. After the PCR amplification, double enzymatic cleavages, enzyme linkage and transformation into E. coli and Mycobacterium smegmatis strains, the enzymatic cleavage maps of the recombinant plasmids is shown in FIG. 14.

RFP MICs of Mycobacterium smegmatis recombinant strains containing pMV261::Rv2629 and pMV261::Rv2629M are detected by absolute concentration method and the results are shown in table 4.

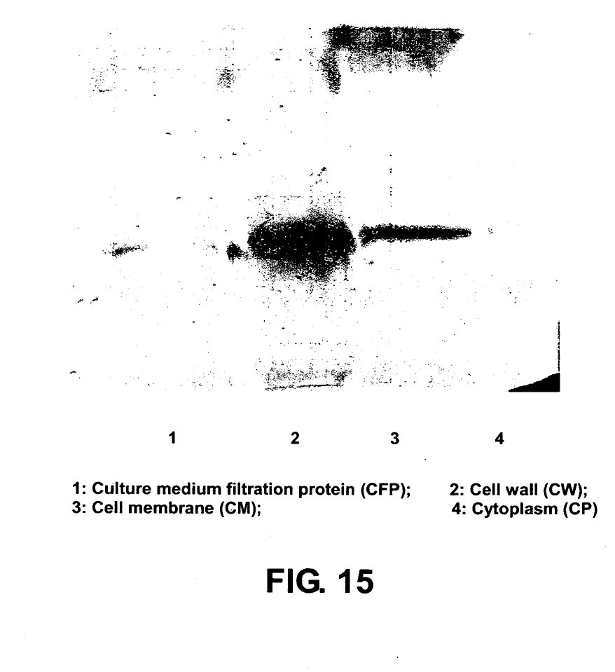

In example 7, the recombinant protein is purified by immunoaffinity columns, and the polyclonal antibody is prepared in Newzealand white rabbits and the potency is detected by ELISA. The isolations of strain subcellular components are carried out according to Kwasi G et al, (Mycobacterium tuberculosis Functional Network Analysis by Global Subcellular Protein Profiling. Molecular Biology of the Cell. 2005, 16, 396-404). Subcellular localization of Rv2629 is carried out by Western-blot technique with 1:1000 self-made rabbit antibody, 1:2000 AP-marked horse anti-rabbit serum and BCIP-NBT from SIGMA as chromogcnic substrate. The result is shown in FIG. 15.

TABLE 2
Characteristics of gene mutations in clinical isolates

| | | | Base change | | No. of RIF^{r} | No. of RIF^{s} |
|---|---|---|---|---|---|---|
| No. | Protein | Locus tag | H37Rv→ clinical | Amino acid change | isolates | isolates |
| R1 | Ssb | Rv0054 | no | no | 13 | 9 |
| R2 | Hypothetical protein | Rv0068 | GTG→CTG | Val(214)→Leu(214) | 3 | 5 |
| R3 | Hypothetical protein | Rv0927 | GCA absent | Ser(140) absent | 13 | 9 |
| R4 | BfrB | Rv3841 | CTA→CTG | Leu(156) →Leu(156) | 5 | 0 |
| | | | ATC→ACC | Ile(157) →Thr(157) | 0 | 4 |
| R5 | OpcA | Rv1446 | GAT→GAA | Asp(70)→Glu(70) | 1 | 0 |
| | | | CGA→CCA | Arg(192)→Pro(192) | 9 | 6 |
| R6 | Antigen 84 | Rv2145 | no | no | 13 | 9 |
| 7# | Hypothetical protein | Rv2159 | no | no | 13 | 9 |
| R8 | CysK | Rv2334 | no | no | 13 | 9 |
| R9 | Hypothetical protein | Rv2629 | GAT→GCT | Asp(64)→Ala(64) | 12 | 5 |
| | | | CGT→CAT | Arg(346)→His(346) | 1 | 0 |
| | | | GAC→CAC | Asp(348) →His(348) | 1 | 0 |
| | | | CTG→TTG | Leu(282)→Leu(282) | 0 | 8 |
| 10# | ilvN | Rv3002 | TCG→TCT | Ser(35) →Ser(35) | 0 | 2 |
| | | | GTG→GGG | Val(52)→Gly(52) | 0 | 0 |
| | | | GAG→AAG | Glu(135)→Lys(135) | 6 | 2 |
| | | | GAG→AAG | Glu(142)→Lys(142) | 3 | 0 |
| R11 | DevR | Rv3133c | no | no | 13 | 9 |
| R12 | PPE | Rv3136 | no | no | 13 | 9 |
| 13# | nuoB | Rv3146 | no | no | 13 | 9 |

TABLE 3
mutations of rpoB in clinical isolates

| | | | | No. of RIF-resistant strains with | | |
|---|---|---|---|---|---|---|
| | | | | indicated RIF MIC range (μg/mL) | | |
| Mutation Position | Nucleotide | Amino Acid | No. (%) of | 1 ≦ MIC < | | |
| (E. coli Position) | Change | Change | Samples | 16 | 16 ≦ MIC < 64 | 64 ≦ MIC |
| Leu 430(511) | CTG→CCG | Lru→Pro | 3(2.68) | 2 | 1 | |
| Gln432(513) | CAA→CCA | Gln→Pro | 2(1.79) | 1 | 1 | |
| Asp435(516) | GAC→GGC | Asp→Gly | 3(2.68) | 1 | 2 | |
| | GAC→TAC | Asp→Tyr | 1(0.89) | | 1 | |
| | GAC→GTC | Asp→Val | 2(1.79) | | 1 | 1 |
| Ser441(522) | TCG→CCG | Ser→Pro | 3(2.68) | 1 | | 2 |
| His 445(526) | CAC→GAC | His→Asp | 7(6.25) | 1 | 2 | 4 |
| | CAC→CTC | His→Leu | 11(9.82) | 3 | | 8 |
| | CAC→CGC | His→Arg | 4(3.57) | 2 | 1 | 1 |
| | CAC→AAC | His→Asn | 3(2.68) | 2 | | 1 |
| | CAC→TAC | His→Tyr | 9(8.04) | 2 | 1 | 6 |
| Ser450(531) | TCG→TTG | Ser→Leu | 33(29.46) | 5 | 10 | 18 |
| | TCG→TGG | Ser→Trp | 15(13.39) | 3 | 2 | 10 |
| | TCG→TAG | Ser→Termination | 2(1.79) | 1 | | 1 |
| Phe424(505), | TTC→TCC, | Phe→Ser, | 2(1.79) | 2 | | |
| Ser450(531) | TCG→TTG | Ser→Leu | | | | |
| Gly426(507), | GGC→GAC, | Gly→Asp, | | | | |
| Asn437(518), | AAC→TAC, | Asn→Tyr, | 1(0.89) | 1 | | |
| Leu457(538) | CTG→CCG | Leu→Pro | | | | |
| None | Wild type | | 11(9.82) | 3 | 5 | 3 |

TABLE 4
MICs of Mycobacterium smegmatis recombinant strains containing
pMV261::Rv2629 and pMV261::Rv2629M

| Recombinant Plasmid | Host strain | MIC (μg/mL) |
|---|---|---|
| — | DH5a | 50 |
| pET30a::Rv2629 | DH5a | 50 |
| pET30a::Rv2629M | DH5a | 170 |
| — | M. smegmatis | 30 |
| pMV261::Rv2629 | M. smegmatis | 30 |
| pMV261::Rv2629M | M. smegmatis | 150 |

What we claim are:

1. A method of screening the drug-resistance protein of Mycobacterium tuberculosis resistant to Rifampicin (RFP, 3-(4-Methylpiperazinyliminomethyl)-rifamycin), including following steps:
(1) Culturing M.tb strains under the same conditions with drug or without drug, and defining a strain having a MIC of 16 μg/mL as a drug-resistance strain;
(2) Isolating proteins from drug-resistance strains and drug sensitive strains respectively;
(3) Comparing the proteins of a drug-resistance strain with that of a sensitive strain and defining a protein additive or absent in the drug-resistance strain or a protein of which the expression increases by least twice or more, or a protein of which expression reduces by at least a half less as a drug-resistance;
(4) Separating and identifying the drug-resistance protein;
(5) Screening the mutated genes related to the drug-resistance by PCR amplification and DNA sequence analysis;
(6) Subcellularly localizing the protein Rv2629 by gene cloning, in vitro expressions and purifications of wild-type protein Rv2629 and mutant-typeprotein Rv2629 respectively, as well as the preparation of a rabbit polyclonal antibody; and
(7) Transforming the wild-type Rv2629 and the mutant-type Rv2629 into Mycobacterium smegmatis strains respectively, and detecting their RFP MICs.

2. The method as claimed in claim 1, characterized in that only the basic proteins from a drug-resistance strain and a sensitive strain are retained in step (2).

3. The method as claimed in claim 1, characterized in that the strains subjected to the MIC detections in step (1), and strains from which proteins are extracted in step (2) are the M.tb strains in logarithmic growth phase.

4. The method as claimed in claim 1, characterized in that the proteins are isolated by electrophoresis, centrifugal, salting-out, filtration or chromatography in step (2) and step (4).

5. The method as claimed in claim 1, characterized in that the proteins are identified by electrephoregram analysis, protein sequencing, mass spectrum analysis or amino acid composition analysis in step (4).

6. The method as claimed in claim 1, characterized in that the resultant drug-resistance protein Rv2629 in step (4) is taken as a drug target for the design and the development of a novel antituberculotic.

7. The method as claimed in claim 1, characterized in that gene Rv2629 and A/C mutant at nucleotide 191 of it are applied as a target for detection of clinical drug-resistant strains.

8. The method as claimed in claim 1, characterized in that the isolated gene Rv2629 and its A/C mutant at nucleotide 191 in steps (4, 5, 6, 7) are used for the construction of vaccines including DNA containing vaccine, subunit containing vaccine, gene-deletion vaccine and gene recombinant vaccine.

9. The method as claimed in claim 1, characterized in that specific primers are used to recombine both the gene of the wild-type Rv2629 and the gene of the mutant-type Rv2629 into E. coli, and then the protein is purified and the antibody is prepared in step (6).

10. The method as claimed in claim 1, characterized in that the recombinant plasmid is transformed into Mycobacterium smegmatis by electrotransformation technique, and then the MIC of the recombinant strain is detected in step (7).
